(19) United States
(12) Patent Application Publication
(10) Pub. No.: US 2022/0152304 A1
(43) Pub. Date: May 19, 2022

(54) INJECTOR ACCESSORIES

(71) Applicant: AMGEN INC., Thousand Oaks, CA (US)

(72) Inventor: Sheldon B. Moberg, Thousand Oaks, CA (US)

(21) Appl. No.: 17/441,021

(22) PCT Filed: Apr. 23, 2020
(86) PCT No.: PCT/US20/29518

§ 371 Date: Sep. 20, 2021

Related U.S. Application Data
(60) Provisional application No. 62/837,823, filed on Apr. 24, 2019.

(51) Int. Cl. A61M 5/20; A61M 5/00; A61M 5/42

(52) U.S. Cl. CPC A61M 5/20; A61M 5/002; A61M 2005/3139; A61M 2005/2006; A61M 5/42

(57) ABSTRACT

A drug delivery device assembly includes an injector body with a proximal end, a distal end, an outer surface, and a knob projecting from the outer surface. A needle assembly is at least partially disposed within the injector body. The needle assembly includes a syringe barrel containing a medicament and a needle or a cannula. A drive assembly is at least partially disposed within the injector body and operably coupled to the needle assembly to urge the medicament through the needle or cannula. An accessory defines a shell and has a proximal end, a distal end, a first opening at the proximal end sized to receive at least a portion of the injector body, and second opening at the distal end. The accessory surrounds a portion of the injector body when the accessory is coupled with the injector body.

### CROSS-REFERENCE TO RELATED APPLICATION

The priority benefit of U.S. Provisional Patent Application No. 62/837,823, filed Apr. 24, 2019, is claimed, and the entire contents thereof are expressly incorporated herein by reference.

### FIELD OF DISCLOSURE

The present disclosure generally relates to drug delivery devices and methods. More particularly, the present disclosure relates to an accessory for drug delivery devices.

### BACKGROUND

Drugs are administered to treat a variety of conditions and diseases. Autoinjectors (e.g., pen style autoinjectors) and on-body injectors offer several benefits in delivery of medicaments and/or therapeutics. One of the benefits can include simplicity of use, as compared with traditional methods of delivery using, for example, conventional syringes. Autoinjectors may be used to deliver many different drugs with varying viscosities and/or desired volumes.

However, autoinjectors sometimes lack ergonomic features that can provide a comfortable user experience to patients with dexterity challenges. Patients might find the lack of an ergonomic grip uncomfortable and difficult to hold the device steady during the injection. Some patients may have reduced manual dexterity and/or cognitive ability, which may make self-injection of drugs physically demanding and can result in treatment noncompliance. For example, the duration of tolerable injection times for patients using handheld autoinjectors is often limited by the patient's ability to sustainably and comfortably grip and control the device while maintaining a stable placement and orientation of the device on the patient's injection site. Some patients may tend to remove the device prior to completion of injection to determine whether the injection was completed. Additionally, family members may often serve as caregivers, and they may not be familiar with the autoinjector product and may themselves suffer from a loss of sensation, dexterity and/or any other flexing or grasping issues in their hands or bodies when attempting to assist with drug administration.

Another issue with existing autoinjector designs includes instability during injection. In some cases, a user must hold the device steadily and carefully in place throughout the injection process in order to effectively and properly administer the drug. Oftentimes, premature removal of the device from the delivery site can result in an incomplete dosage being delivered due to the drug spraying onto the skin surface. In other cases, a user must stabilize the injection site to properly activate the autoinjector drug delivery. Autoinjectors typically have a feature on the front of the device that acts to unlock or initiate the injection when pressed against a users skin. The reliability of the activation can be dependent upon the condition of the skin or tissue where the drug is injected where less stable tissue may result in difficulty with activation. The activation requires a specific force to be applied to the device and that force is transferred to the user's skin. The force is translated into a pressure and, depending upon the surface area of the device that interfaces with the users skin, this pressure must significantly displace the user's tissue until sufficient force has been achieved to unlock or initiate the injection. This can result in requiring additional forceful application of the autoinjector onto the injection site, which may cause significant discomfort and hesitation in administering the medicament in the patient.

As described in more detail below, the present disclosure sets forth an accessory for drug delivery devices, such as autoinjectors, that embodies advantageous alternatives to existing systems and methods, and that may address one or more of the challenges or needs mentioned herein, as well as provide other benefits and advantages.

### SUMMARY

In accordance with a first exemplary aspect, a drug delivery device assembly may include an injector and an accessory. The injector may include an injector housing having a body with a proximal end, a distal end, and a longitudinal axis extending between the proximal end and the distal end. A needle assembly may be at least partially disposed within the body adjacent to the proximal end. The needle assembly may include a syringe barrel containing a medicament and a needle or a cannula. A drive assembly may be at least partially disposed within the body and operably coupled with the needle assembly to urge the medicament through the needle or cannula. The accessory may define a shell having a proximal end, a distal end, and a longitudinal axis extending between the proximal end and the distal end. The proximal end of the shell may include a first opening sized to receive a portion of the injector. The distal end of the shell may include a second opening. The longitudinal axis of the body may be generally aligned with the longitudinal axis of the shell when the shell is coupled with the body of the injector housing.

In accordance with a second exemplary aspect, a drug delivery device assembly may include an injector body with a proximal end, a distal end, and an outer surface. A needle assembly may be at least partially disposed within the injector body and may include a syringe barrel containing a medicament and a needle or a cannula. A drive assembly may be at least partially disposed within the injector body and operably coupled with the needle assembly to urge the medicament through the needle or cannula. An accessory may define a shell with a proximal end, a distal end, a first opening at the proximal end sized to receive at least a portion of the injector body, a second opening at the distal end, and a cut-out or an indention formed in the shell and configured to secure the accessory and the injector body.

In accordance with a third exemplary aspect, an accessory for a drug delivery device may include a shell with a proximal end, a distal end, and a longitudinal axis extending between the proximal end and distal end. The accessory may include a first opening at the proximal end, a second opening at the distal end, and a throughbore connecting the first opening and the second opening. The throughbore may be sized to receive a portion of a drug delivery device. Further, the accessory may include at least one of (a) and (b), (a) a plurality of fins extending radially outward from the proximal end, and (b) a cut-out formed in the shell and disposed between the first opening and the second opening.

In further accordance with any one or more of the foregoing first, second, or third aspects, a drug delivery device assembly and an accessory for a drug delivery device may further include any one or more of the following preferred forms.

In a preferred form, the body may include an outer surface and a knob projecting from the outer surface.

In a preferred form, the accessory may include a debossed feature disposed on an interior surface of the shell.

In a preferred form, the debossed feature may be shaped to slidably receive the knob of the body.

In a preferred form, the debossed feature may extend from an edge of the proximal end of the shell to a notch formed in the interior surface of the shell.

In a preferred form, a width of the debossed feature may narrow from the edge of the proximal end of the shell to the notch.

In a preferred form, the notch may be sized to receive the knob of the body of the injector housing to couple the injector housing with the shell.

In a preferred form, the shell may include at least one cut-out that is aligned with a dosage window of the injector.

In a preferred form, the shell may have a corrugated outer surface.

In a preferred form, the accessory may include a plurality of fins extending radially outward from the proximal end of the shell.

In a preferred form, the first opening may be greater than the second opening.

In a preferred form, the second opening may be adjacent to the proximal end of the shell and sized to permit the needle or cannula to pass through.

In a preferred form, the shell may include a ledge adjacent to the second opening.

In a preferred form, the ledge extending radially inward relative to the longitudinal axis of the shell

In a preferred form, when the accessory is coupled with the injector, the ledge of the shell may be adjacent to a planar surface of the proximal end of the injector.

In a preferred form, the accessory may include a flange extending radially outward relative to the longitudinal axis.

In a preferred form, the flange may be disposed at the proximal end of the shell.

In a preferred form, an indentation may be formed on an interior surface of the shell.

In a preferred form, the indentation may be shaped to slidably receive a knob projecting from an outer surface of the injector body.

In a preferred form, the indentation of the shell may extend from an edge of the proximal end of the shell to a notch formed at a location spaced from the edge of the proximal end of the shell.

In a preferred form, the notch may be sized to receive the knob of the injector body to couple the injector body with the shell by a snap-fit connection.

In a preferred form, the notch may be spaced away from a cut-out.

In a preferred form, the indentation may be at least partially defined by an angled wall.

In a preferred form, the angled wall may be arranged to guide a portion projecting from an outer surface of the injector body into a notch formed at a location spaced from an edge of the proximal end of the shell.

In a preferred form, a cut-out may be formed in the shell and at least partially surrounded by the indentation formed in the interior surface of the shell.

In a preferred form, the cut-out or indentation may be generally triangular to help guide a portion of the injector body to a notch adjacent to the cut-out or indentation.

In a preferred form, an indentation may be formed on an interior surface of the shell and may surround the cut-out formed in the shell.

In a preferred form, the indentation may be adapted to receive a portion projecting from an outer surface of the drug delivery device.

In a preferred form, the indentation of the shell may extend from an edge of the proximal end of the shell to a notch formed at a location spaced from the edge of the proximal end of the shell.

In a preferred form, the notch may be adapted to receive the portion projecting from the outer surface of the drug delivery device by a snap-fit connection.

In a preferred form, the shell may include a ledge extending radially inward relative to the longitudinal axis of the shell and defining the second opening.

In a preferred form, the second opening may be smaller than the first opening.

### BRIEF DESCRIPTION OF THE DRAWINGS

The above needs are at least partially met through provision of the drug delivery device having an accessory described in the following detailed description, particularly when studied in conjunction with the drawings, wherein:

FIG. 1 is a side view of an example injector assembled in accordance with the teachings of the present disclosure;

FIG. 2 is a perspective view of a first exemplary injector accessory assembled in accordance with the teachings of the present disclosure;

FIG. 3 is an exploded perspective view of the injector of FIG. 1 and the first exemplary injector accessory of FIG. 2;

FIG. 4 is perspective view of a drug delivery device assembly including the injector of FIG. 1 assembled with the first exemplary injector accessory of FIG. 2;

FIG. 5 is a perspective view of a second exemplary injector accessory assembled in accordance with the teachings of the present disclosure;

FIG. 6 is a different perspective view of the second exemplary injector accessory of FIG. 5;

FIG. 7 is a perspective view of a third exemplary injector accessory assembled in accordance with the teachings of the present disclosure;

FIG. 8 is a different perspective view of the third exemplary injector accessory of FIG. 7;

FIG. 9 is a perspective front view of a fourth exemplary injector accessory assembled in accordance with the teachings of the present disclosure;

FIG. 10 is a perspective back view of the fourth exemplary injector accessory of FIG. 9;

FIG. 11 is a front view of the fourth exemplary injector accessory of FIG. 9; and

FIG. 12 is a perspective view of a second exemplary drug delivery device assembly including the injector of FIG. 1 and the fourth exemplary injector accessory of FIG. 9.

### DETAILED DESCRIPTION

Generally speaking, pursuant to these various embodiments, a drug delivery device (e.g., an autoinjector or other injector) is coupled to an accessory that at least partially surrounds the injector to improve the injector grip, customize the appearance of the injector, and/or to improve or facilitate injection into the patient.

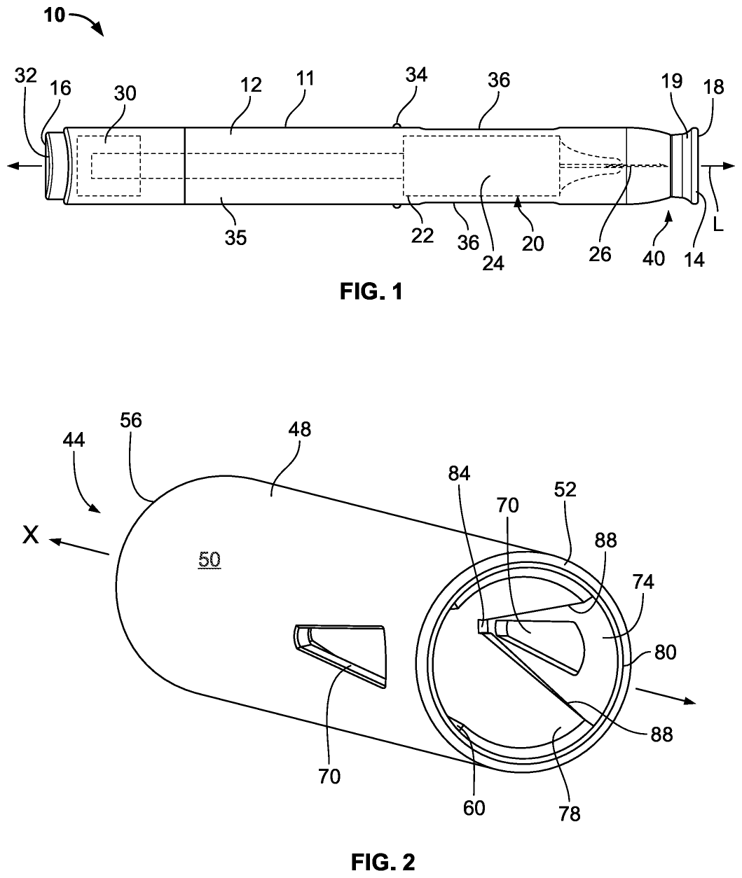

As illustrated in FIG. 1, an example injector 10 generally includes an injector housing 11 defining a body 12 that includes a proximal end 14, a distal end 16, and a longitudinal axis L extending between the proximal and distal ends 14, 16. The body 12 further includes a generally planar contact surface 18 of a needle shield 19 positioned at the proximal end 14. A needle assembly 20 is at least partially disposed within the body 12 adjacent to or near the proximal end 14, and includes a syringe barrel 22 that contains a medicament 24 and a needle or a cannula 26 that is used to inject the medicament 24 into a patient. In the illustrated example, the needle or cannula 26 is initially positioned within the body 12 prior to activation, and may protrude through an opening in the proximal end 14 during drug delivery.

A drive assembly 30 is also at least partially disposed within the body 12 and is operably coupled with the needle assembly 20. The drive assembly 30 may include an actuator button 32 positioned at or near the distal end 16 of the body 12 that initiates actuation of the drive assembly 30. In operation, a user places the contact surface 18 of the needle shield 19 of the body 12 against their skin (e.g., on their leg or their stomach) and actuates the actuator button 32. This actuation causes a drive mechanism (in the form of a spring, a motor, a hydraulic or pressurized mechanism, etc.) of the drive assembly 30 to exert a driving force on the needle assembly 20 that causes the needle or cannula 26 to be inserted through the opening of the body 12 and into a patient, and that further causes the medicament 24 to be urged from the syringe barrel 22, out the needle or cannula 26, and into the patient. In some versions, the patient may manually insert the needle or cannula 26, and actuation of the drive mechanism 30 only includes causing the medicament 24 to be urged from the syringe barrel 22, out the needle or cannula 26, and to the patient.

The injector 10 may include any number of additional features and components that may assist and/or enhance the functionality of the device. In the illustrated example, one or more knobs 34 project from an outer surface 35 of the body 12 as an anti-roll feature. The one or more knobs 34 may be integrally formed with the body 12 of the injector 10 or may be coupled to the outer surface 35 by welding, adhesive, or by another adhering method. Additionally, a dosage window 36 positioned at or near the syringe barrel 22 provides a visual indication of the remaining quantity of drug during administration. The injector 10 also includes a protective assembly 40 partially disposed within the housing 11 at the proximal end 14 of the body 12 to shield the needle 26 and prevent unintentional activation of the injector 10 and deployment of the needle or cannula 26. The protective assembly 40 acts to unlock or initiate the injection when the planar surface 18 is pressed against a users skin. The activation of the protective assembly 40 requires a specific force to be applied to the planar surface 18 of the injector 10 and that force is transferred to the users skin. In other examples, the injector 10 may additionally include one or more electronic modules that are coupled to the body 12, the needle assembly 20, the drive assembly 30, and/or any other components of the injector 10. Further, the injector 10 may also include any number of safety mechanisms such as the needle shield 19, retraction mechanism, damping mechanism, and the like.

The present example of the drug delivery device 10 takes the form of an autoinjector or pen-type injector, and, as such, may be held in the hand of the user over the duration of drug delivery. The drug delivery device 10 is operable for self-administration by a patient or for administration by caregiver or a formally trained healthcare provider (e.g., a doctor or nurse). However, various implementations and configurations of the drug delivery device 10 are possible. In other examples, the drug delivery device 10 may be configured as a multiple-use reusable injector.

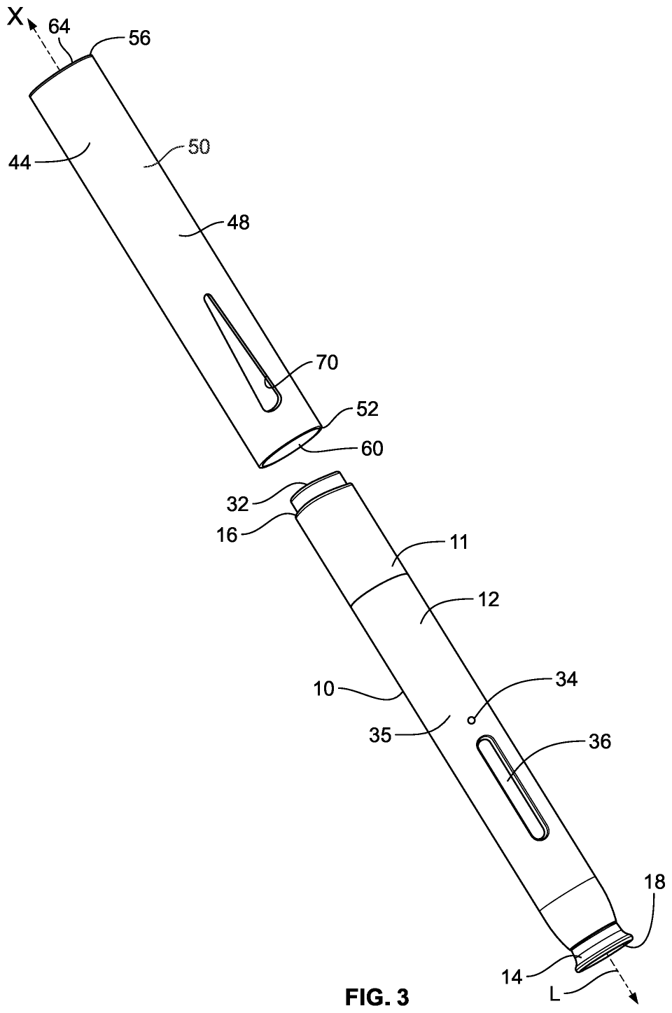

In FIG. 2, a first exemplary accessory 44 for a drug delivery device, such as the injector 10 of FIG. 1, is constructed in accordance with the teachings of the present disclosure. The accessory 44 defines a shell 48 having a proximal end 52, a distal end 56, and a longitudinal axis X extending between the proximal and distal ends 52, 56. The shell 48 includes a first opening 60 disposed at the proximal end 52 and sized to receive a first portion, and specifically the distal end 16, of the body 12 of the injector 10. The shell 48 includes a second opening 64 (as shown in FIG. 3) at the distal end 56 and opposite the first opening 60 and is sized to receive a second portion of the injector 10. The shell 48 is cylindrical with a bore connecting the first and second openings 60, 64.

The shell 48 includes one or more cut-outs 70, for example two cut-outs 70, that are configured to align with one or more dosage windows 36 of the injector 10 when the shell 48 is coupled with the injector body 12. Each cut-out 70 is surrounded by a debossed feature 74, or indentation 74, formed on an inner surface 78 of the shell 48. The debossed feature 74 is triangular or tear-drop shaped such that a width of the indentation 74 gradually narrows from an edge 80 of the proximal end 52 of the shell 48 to a location spaced away from the edge 80. In particular, the debossed feature 78 extends between the edge 80 of the proximal end 52 to a notch 84 formed on the inner surface 78 of the shell 48. When the shell 48 is coupled to the injector body 12, the indentation 74 is aligned with the dosage window 36 of the injector 10.

The notch 84 of the indentation 74 is sized to receive one of the knobs 34 protruding from the outer surface 35 of the injector body 12. The shape of the indentation 74 surrounding the cut-out 70 is configured to guide the knobs 34 of the injector 10 into alignment with the notches 84 of the indentations 74 so that each knob 34 snaps into one notch 84 when the shell 48 is properly in place relative to the injector body 12. The indentation 74 defines two sloped barriers 88 or walls that assist in directing the knobs 34 into alignment with the notches 84. If the knob 34 is out of alignment with the notch 84, the knob 34 will slide along one of the barriers 88, slightly rotating relative to the shell 48, until the notch 84 receives the knob 34 by interference fit. In other examples, the indentation 74 may be different in size and shape than what is illustrated, and may be, for example, an oval or rectangular slot. In some examples, the shell 48 may include an indentation 74 that does not surround the cut-out 70, but that is arranged to couple the shell 48 with the body 12 of the injector 10. The notch 84 is located relative to the shell 48 to ensure that the shell 48 couples to the injector 10 at a particular location.

The notch 84 may be rounded or have a square shape at an end of the indentation 74, and may be shaped specifically to receive a portion of the drug delivery device, and specifically, to receive the knob 34 of the injector 10. In some other examples, the indentation or cut-out of the shell 48 may guide the knob 34 along a track that extends longitudinally and circumferentially to couple the shell 48 to the injector 10. In that case, to couple the injector 10 to the shell 48, the shell must be pushed onto the proximal end 14 of the injector housing 12 a certain distance and then rotated so slide the knob 34 or other portion of the injector 10 into the notch 84.

In FIG. 3, the longitudinal axis L of the injector 10 is aligned with the longitudinal axis X of the shell 48 for inserting the injector 10 into the shell 48. To insert the injector 10 into the accessory 44, the proximal end 52 of the shell 48 is initially placed above or adjacent to the distal end 16 of the injector 10 as shown in FIG. 3. The button 32 of the injector 10 slides through the first opening 60 of the accessory 10 during insertion. The triangular debossed features 74 on the inner surface 78 of the shell 48 guide the injector 10 into a correct position to align the cutouts 70 on the shell 48 with one or more windows 36 of the injector 10. Put differently, the indentations 74 slidably receive the knobs 34 of the injector body 12 as the injector 10 slides into place within the shell 48. If the injector 10 is out of alignment with the shell 48 during insertion, then the protruding knob 34 on the outer surface 35 of the injector body 12 will slide against the angled wall of the sloped barrier 88 defined by the debossed feature 74 until the knob 34 slides into the notch 84 of the shell 48. As the knob 34 slides against the sloped barrier 88, the injector 10 or the shell 48 rotates about the longitudinal axis L or X until the knob 34 is coupled to the notch 84. Once the injector 10 is fully inserted into the shell 48 of the accessory 44, the coupling between the notch 84 and knob 34 may effectively inhibit the injector 10 from breaking free from or rotating within the shell 48. In the illustrated example, the one or more knobs 34 are hemispherical protrusions and the indentations 74 are triangular or tear-drop shaped. However, in other examples, the indentations 74 may have a different geometery, such as, for example, rectangular, oval, etc., and the knobs 34 maybe cubic, pointed, ridged, or have a different geometry to facilitate sliding within the indentations 74 of the shell 48.

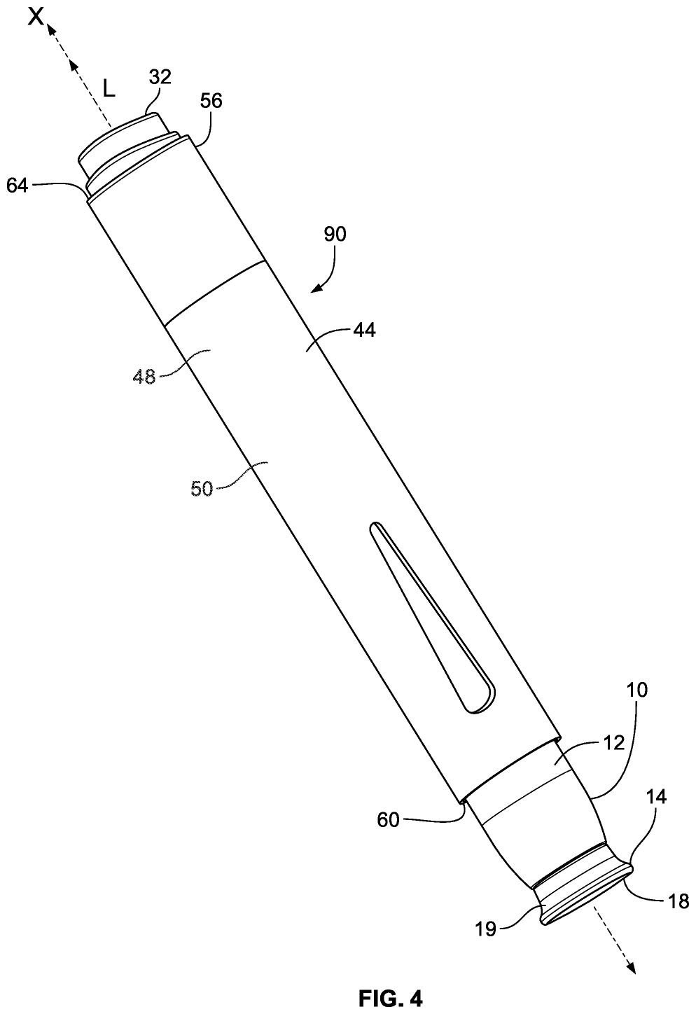

As shown in FIG. 4, a drug delivery device assembly 90 is shown with the injector 10 coupled to the accessory 44. When assembled, the longitudinal axis L of the injector 10 is generally aligned with the longitudinal axis X of the accessory 44 and the shell 48 is coupled with the body 12 of the injector housing 11. The proximal end 14 of the injector 10 extends from the first opening 60 at the proximal end 52 of the shell 48 and the button 32 at the distal end 16 of the injector body 12 extends from the second opening 62 at the distal end 56 of the shell 48. In this way, the accessory 44 does not interfere with the operation of the injector 10 (i.e., pressing the button 32 and activating the protective assembly 40) while also surrounding most of the outer surface 35 of the injector body 12.

The shell 48 may be designed or decorated to change the appearance and the grip of the injector 10 completely, while permitting a user to view the level of remaining medicament during injection. For example, while the shell 48 of the accessory 44 is cylindrical and has a generally smooth outer surface 50, other exemplary accessories may include corrugated, textured, undulating surface treatments to enhance the ergonomics and style of the assembly 90. The outer surface 50 of the shell 48 can be decorated with decals or labels based on patient preference for further personalization. The shell 48 may have ergonomic grip improving features such as, for example, a ribbed surface, stippling pattern, knurling pattern and/or a flange that provides leverage and control for the patient's hand.

While the exemplary injector 10 is disposable, the accessory 44 is reusable and may be easily removed from one injector body 12 by pulling the shell 48 away from the injector body 12. To remove a used injector from the accessory 44, a user may pull the shell 48 such that the proximal end 52 of the shell 44 moves toward the proximal end 14 of the injector body 12 to separate the one or more knobs 34 of the injector 10 from the one or more notches 84 of the shell 48. In another method, a user may place planar surface 18 onto a flat surface and slide the shell 48 of the accessory 44 toward the flat surface, thereby separating the one or more knobs 34 from the one or more notches 84 to decouple the accessory 44 from the injector 10.

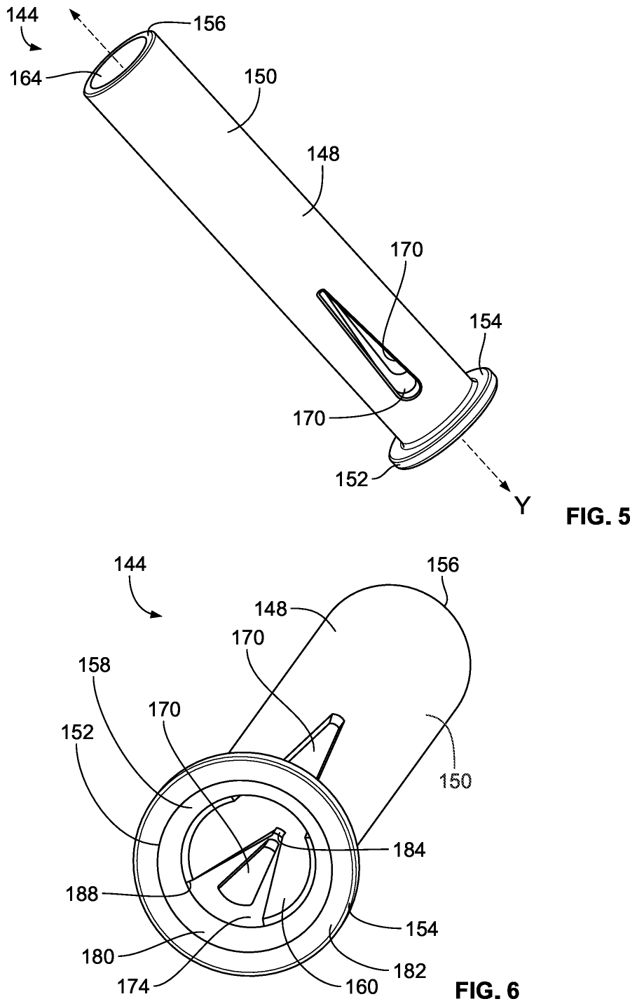

FIGS. 5 and 6 illustrate a second exemplary accessory 144 constructed in accordance with the teachings of the present disclosure. The second exemplary accessory 144 is similar to the first exemplary accessory 44 of FIGS. 2-4, and may be coupled to a drug delivery device, like the injector 10 of FIG. 1. Thus, for ease of reference, and to the extent possible, the same or similar components of the second exemplary accessory 144 will retain the same reference numbers as outlined above with respect to the first exemplary accessory 44, although the reference numbers will be increased by 100. However, the second exemplary accessory 144 differs from the first exemplary accessory 44 in the manner described below.

Like the first exemplary accessory 44, the second exemplary accessory 144 is generally cylindrical with a bore connecting a first opening 160 and a second opening 164. At a proximal end 152 of a shell 148 of the accessory 144, a flange 154 extends radially outwardly relative to a longitudinal axis Y of the shell 140. In particular, the flange 154 protrudes away from an edge 180 of the shell 148. The flange 154 may extend both radially and axially relative to the longitudinal axis Y, thereby forming a sloped interior surface 158 that connects the edge 180 and a planar surface 182 of the proximal end 152. The flange 154 may beneficially provide a gripping feature to the accessory 144. Additionally, the flange 154 is arranged to spread and stretch the skin of a patient outward to help flatten or tighten the injection point to facilitate injection and to firm up the injection site.

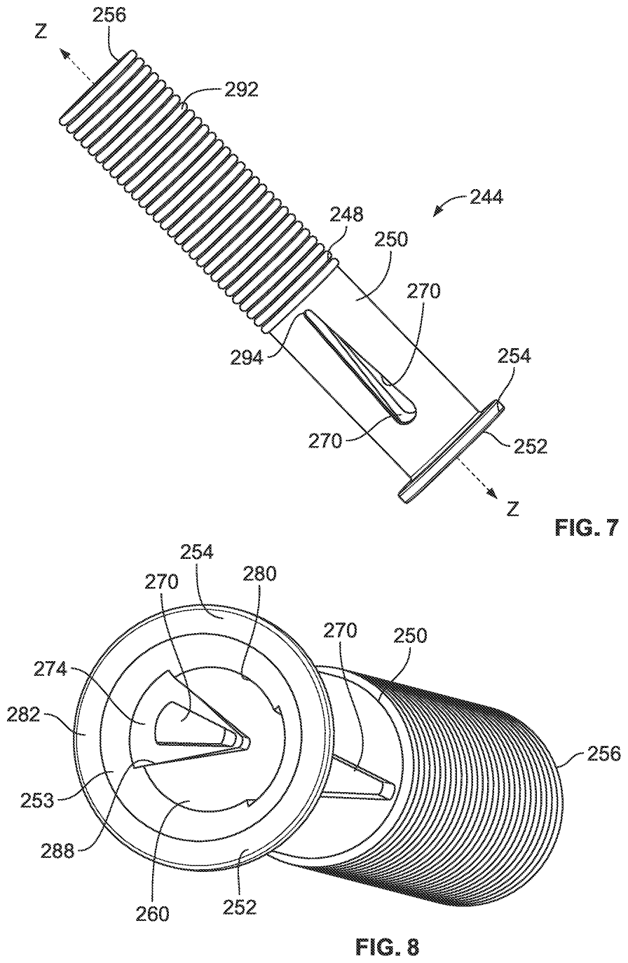

FIGS. 7 and 8 illustrate a third exemplary accessory 244 constructed in accordance with the teachings of the present disclosure. The third exemplary accessory 244 is similar to the first exemplary accessory 44 of FIGS. 2-4, and may be coupled to a drug delivery device, like the injector 10 of FIG. 1. Thus, for ease of reference, and to the extent possible, the same or similar components of the third exemplary accessory 244 will retain the same reference numbers as outlined above with respect to the first exemplary accessory 44, although the reference numbers will be increased by 200. However, the third exemplary accessory 244 differs from the first exemplary accessory 44 in the manner described below.

Like the first exemplary accessory 44, the third exemplary accessory 244 is generally cylindrical with a bore connecting a first opening 260 and a second opening (hidden from view). At a proximal end 252 of a shell 248 of the accessory 244, a flange 254 extends radially outwardly relative to a longitudinal axis Z of the shell 240. In particular, the flange 254 protrudes away from an edge 280 of the shell 248. The flange 254 may extend both radially and axially relative to the longitudinal axis Z, thereby forming a sloped interior surface 258 that connects the edge 280 and a planar surface 282 of the proximal end 252. The flange 254 may beneficially provide a gripping feature to the accessory 244. In addition to the flange 254, a portion of the shell 248 includes a corrugated surface 292. The corrugated surface 292 of the accessory 244 shown in FIGS. 7 and 8 includes a pattern of ridges and grooves extending from a distal end 256 to a location adjacent to a tip 294 of one or more cutouts 270 in the shell 248. However, in other examples, the corrugated surface 292 may be a spiraled ridge, groove, or a plurality of dimples, bumps, or curves. Additionally, the corrugated surface 292 may extend entirely along a length of the shell 248 from the distal end 256 to the proximal end 254, or may partially extend along the length of the shell 248 in patches or in localized areas, such as, for example, where a user typically grips the injector 10.

The accessories 44, 144, 244 are preferably made of a durable polymer, such as polyethylene, that may be formed by injection molding, thermoforming, or compression molding. In other examples, the accessories 44, 144, 244 may instead be formed of any other suitable and durable material including metal, fiberglass, or other similar materials, or any combination of these materials. The shell 48, 148, 248 of each accessory 44, 144, 244 may include a surface treatment to permit a user to decorate and personalize the drug delivery device assembly 90 and may include features to improve upon the ergonomics of the grip of the injector 10. The patient's ability to accessorize or personalize their delivery device may help to enhance their experience by providing differentiation that speaks to their unique style and personality and can help devices appear less like sterile medical devices. Further, providing ergonomic features allows for a more stress free and comfortable user experience while preventing injection errors for patients with reduced dexterity.

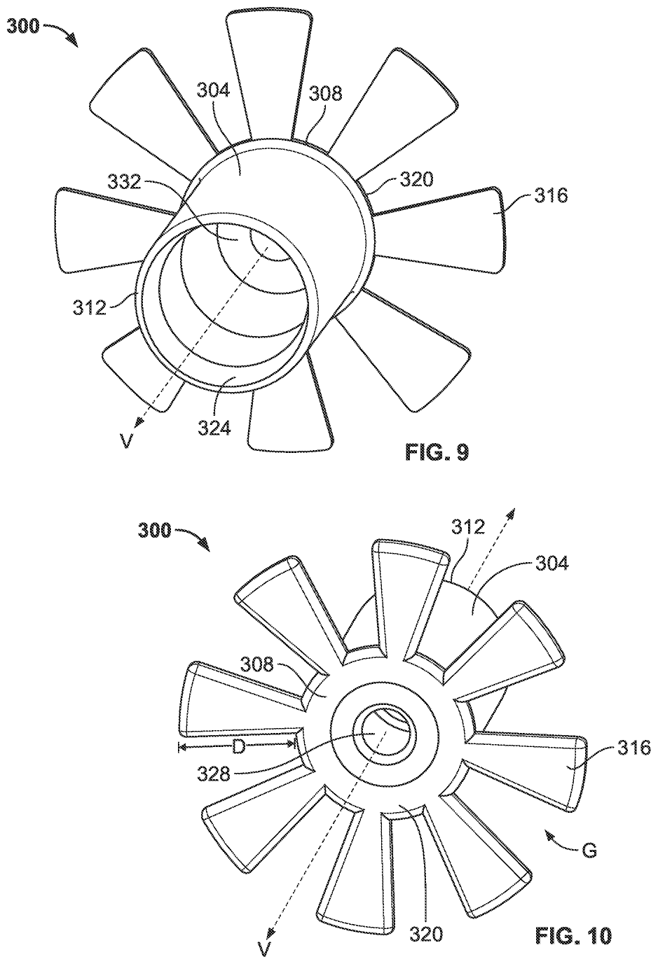

Turning now to FIGS. 9-12, a fourth exemplary accessory 300 is constructed in accordance with the teachings of the present disclosure. The fourth exemplary accessory 300 is arranged to attach to a proximal end of a drug delivery device, such as the proximal end 14 the injector 10, as shown in FIG. 12. The accessory 300 facilitates activation of the protective assembly 40 of the injector 10 by ensuring that the injection site is secure enough for injection. As with the first, second, and third exemplary accessories 44, 144, 244 described above, the fourth exemplary accessory 300 may be reused with multiple injectors and can easily be coupled and decoupled to the injector 10 for use.

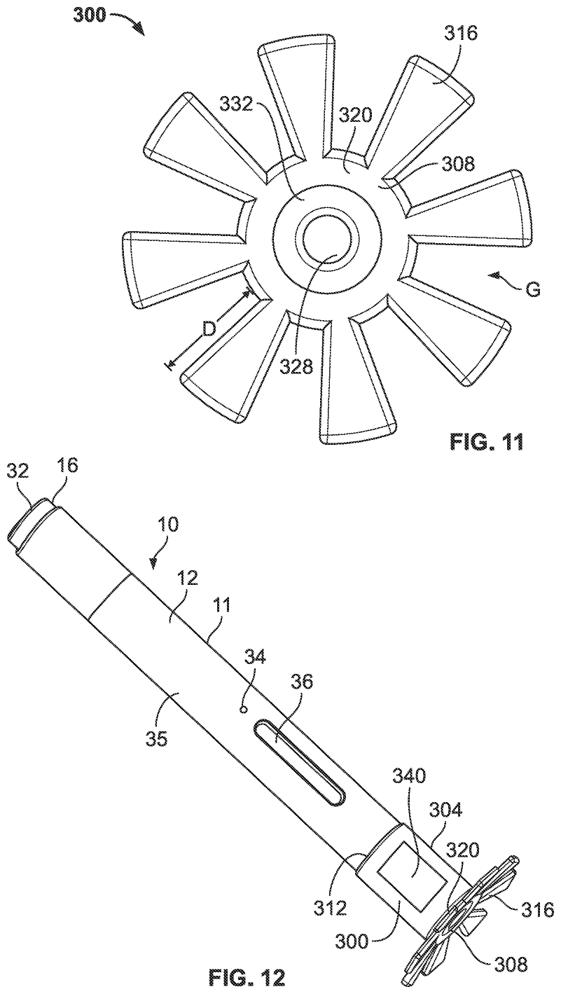

As shown in FIGS. 9-11, the accessory 300 includes a cylindrical shell 304 with a proximal end 308, a distal end 312, and a longitudinal axis V extending between the proximal and distal ends 308, 312. At the proximal end 308, a plurality of fins 316 extends radially outward from a base 320 of the shell 304 and relative to the longitudinal axis V. A first opening 324 is formed in the distal end 312 of the shell 304, and as shown in FIGS. 10 and 11, a second opening 328 is defined in the proximal end 308 of the shell 304. The first opening 324 is sized to receive the proximal end 14 of the injector body 12, and the second opening 328 is smaller than the first opening 324 but wide enough to permit the needle or cannula 26 to pass through during injection. As shown in FIG. 11, the second opening 328 is defined by a ledge 332 that extends radially inwardly from the shell 304 and relative to the longitudinal axis V. The ledge 332 is configured to interface with the planar surface 18 of the needle shield 19 of the injector 10 to disburse the force applied to the injector 10 by the user to unlock or initiate injection into the patient. When the accessory 300 is coupled to the injector 10, the planar surface 18 of the injector 10 sits behind the ledge 332 of the accessory 300.

As shown in FIGS. 10 and 11, eight fins 316 extend from the base 320 and are equally spaced relative to each other. The fins 316 are configured to stretch the skin of a patient when the injector 10 is pressed against the injection site. Each fin 316 extends from the base a distance D and are trapezoidal such that a width of each fin 316 is wider away from the base 320 and narrower at the location where each fin 316 meets the base 320. Each fin 316 is equidistant from adjacent fins 316 and is separated from adjacent fins by a gap G. However, in other examples, the accessory 300 may include more or fewer than eight fins 316 and the fins may have a different geometry, such as, for example, petal shaped, oval, circular, triangular, etc.

As previously mentioned, the protective assembly 40 of the injector 10 may be activated when the planar surface 18 of the injector 10 is pressed against the skin of the patient. The reliability of the activation of the protective assembly 40 may be dependent upon the condition of the skin or tissue where the drug is injected. For example, weaker tissue may result in difficulty with activation and may require more force against the patient's skin to activate injection. The activation requires a specific force to be applied to the device 10 and that force is transferred to the user's skin as applied pressure. The plurality of fins 316 of the accessory 300 advantageously increases the contact surface area at the injection site so that the applied pressure can displace the users tissue to unlock or initiate the injection with minimal amount of force applied. The fins 316 provide a wider base than what is traditionally offered by the planar surface 18 of the injector 10 and provide stabilization when stretched horizontally as the patient presses the injector 10 against the injection site. Specifically, the fins 316 flex against the skin, effectively disbursing the applied force over a greater surface area which minimizes the displacement of the tissue and force required to initiate injection.

The shell 304 may be designed or decorated to change the appearance and the grip of the injector 10 completely, while permitting a user to view the level of remaining medicament during injection. For example, while the shell 304 of the accessory 300 is cylindrical and has a generally smooth outer surface, other exemplary accessories may include corrugated, textured, undulating surface treatments to enhance the ergonomics and style of the assembly 350 of FIG. 12. The outer surface of the shell 304 can be decorated with decals or labels based on patient preference for further personalization. The shell 304 may have ergonomic grip improving features such as, for example, a ribbed surface, stippling pattern, knurling pattern and/or a flange that provides leverage and control for the patient's hand.

An additional visual indication may be provided to inform the user that the needle shield is fully compressed. For example, the accessory 300 may include a window 340, as illustrated in FIG. 12, aligned with the needle shield to permit a user to view the compression of the needle shield while the user is using the injector 10. In another example, the shell 304 of the accessory 300 may be a transparent material so that the proximal end 14 of the injector 10 can be substantially or entirely viewed when the accessory 300 is attached to the injector 10.

The accessory 300 is preferably made of a durable polymer, such as polyethylene, that may be formed by injection molding, thermoforming, or compression molding. The stabilizer is made from a flexible polymeric material that contains appropriate shape memory to provide stability to the patient when pressed against the injection site. In other examples, the accessory 300 may instead be formed of any other suitable and durable material including metal, fiberglass, or other similar materials, or any combination of these materials.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“ID-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4I37 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-?4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFN? mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFR mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

1. A drug delivery device assembly comprising:
an injector including:
an injector housing having a body with a proximal end, a distal end, and a longitudinal axis extending between the proximal end and the distal end;
a needle assembly at least partially disposed within the body adjacent to the proximal end, the needle assembly comprising a syringe barrel containing a medicament and a needle or a cannula;
a drive assembly at least partially disposed within the body and operably coupled with the needle assembly to urge the medicament through the needle or cannula; and
an accessory defining a shell having a proximal end, a distal end, and a longitudinal axis extending between the proximal end and the distal end, the proximal end of the shell including a first opening sized to receive a portion of the injector, the distal end of the shell including a second opening;
wherein the longitudinal axis of the body is generally aligned with the longitudinal axis of the shell when the shell is coupled with the body of the injector housing.

2. The assembly of claim 1, wherein the body includes an outer surface and a knob projecting from the outer surface, and wherein the accessory includes a debossed feature disposed on an interior surface of the shell, the debossed feature being shaped to slidably receive the knob of the body.

3. The assembly of claim 2, wherein the debossed feature extends from an edge of the proximal end of the shell to a notch formed in the interior surface of the shell.

4. The assembly of claim 3, wherein a width of the debossed feature narrows from the edge of the proximal end of the shell to the notch.

5. The assembly of claim 4, wherein the notch is sized to receive the knob of the body of the injector housing to couple the injector housing with the shell.

6. The assembly of claim 4, wherein the shell further includes at least one cut-out that is aligned with a dosage window of the injector, and/or (b) a corrugated outer surface.

7. (canceled)

8. The assembly of claim 1, wherein the accessory includes a plurality of fins extending radially outward from the proximal end of the shell.

9. The assembly of claim 1, wherein the first opening is greater than the second opening, the second opening adjacent to the proximal end of the shell and sized to permit the needle or cannula to pass through.

10. The assembly of claim 1, wherein the shell includes a ledge adjacent to the second opening, the ledge extending radially inward relative to the longitudinal axis of the shell, wherein when the accessory is coupled with the injector, the ledge of the shell is optionally adjacent to a planar surface of the proximal end of the injector.

11. (canceled)

12. The assembly of claim 1, wherein the accessory includes a flange extending radially outward relative to the longitudinal axis, the flange disposed at the proximal end of the shell.

13. A drug delivery device assembly comprising:
an injector body with a proximal end, a distal end, and an outer surface;
a needle assembly at least partially disposed within the injector body, the needle assembly including a syringe barrel containing a medicament and a needle or a cannula;
a drive assembly at least partially disposed within the injector body and operably coupled with the needle assembly to urge the medicament through the needle or cannula; and
an accessory defining a shell with a proximal end, a distal end, a first opening at the proximal end sized to receive at least a portion of the injector body, a second opening at the distal end, and a cut-out or an indention formed in the shell and configured to secure the accessory and the injector body.

14. The assembly of claim 13, further comprising an indentation formed on an interior surface of the shell, the indentation shaped to slidably receive a knob projecting from an outer surface of the injector body.

15. The assembly of claim 14, wherein the indentation of the shell optionally extends from an edge of the proximal end of the shell to a notch formed at a location spaced from the edge of the proximal end of the shell.

16. The assembly of claim 15, wherein the notch is at least one of (a) sized to receive the knob of the injector body to couple the injector body with the shell by a snap-fit connection, and/or (b) spaced away from a cut-out.

17. (canceled)

18. The assembly of claim 13, wherein the indentation is at least partially defined by an angled wall, wherein the angled wall is optionally arranged to guide a portion projecting from an outer surface of the injector body into a notch formed at a location spaced from an edge of the proximal end of the shell.

19. (canceled)

20. The assembly of claim 14, further comprising a cut-out formed in the shell and at least partially surrounded by the indentation formed in the interior surface of the shell, wherein optionally the cut-out or indentation is generally triangular to help guide a portion of the injector body to a notch adjacent to the cut-out or indentation.

21. (canceled)

22. An accessory for a drug delivery device, the accessory comprising:
a shell with a proximal end, a distal end, and a longitudinal axis extending between the proximal end and distal end;
a first opening at the proximal end;
a second opening at the distal end;
a throughbore connecting the first opening and the second opening, the throughbore sized to receive a portion of a drug delivery device;
at least one of (a) and (b), (a) a plurality of fins extending radially outward from the proximal end, and (b) a cut-out formed in the shell and disposed between the first opening and the second opening.

23. The accessory of claim 22, further comprising an indentation formed on an interior surface of the shell and surrounding the cut-out formed in the shell, the indentation adapted to receive a portion projecting from an outer surface of the drug delivery device, wherein the indentation of the shell optionally extends from an edge of the proximal end of the shell to a notch formed at a location spaced from the edge of the proximal end of the shell, the notch adapted to receive the portion projecting from the outer surface of the drug delivery device by a snap-fit connection.

24. (canceled)

25. The accessory of claim 22, wherein the shell includes a ledge extending radially inward relative to the longitudinal axis of the shell and defining the second opening, and wherein the second opening is smaller than the first opening, and/or (b) a corrugated outer surface.

26. (canceled)

27. The accessory of claim 22, wherein the accessory includes a flange extending radially outward relative to the longitudinal axis, the flange disposed at the proximal end of the shell.
